(19) United States
(12) Patent Application Publication
(10) Pub. No.: US 2024/0062895 A1
(43) Pub. Date: Feb. 22, 2024

(54) METHOD AND FACILITY FOR PROVIDING A SET OF CONTROL COMMANDS FOR CONTROLLING A MEDICAL IMAGING SYSTEM

(71) Applicant: Siemens Healthcare GmbH, Erlangen (DE)

(72) Inventor: Corinna MAIER-MATIC, Muenchen (DE)

(73) Assignee: Siemens Healthcare GmbH, Erlangen (DE)

(21) Appl. No.: 18/450,585

(22) Filed: Aug. 16, 2023

(30) Foreign Application Priority Data
Aug. 18, 2022 (DE) 10 2022 208 589.2

(51) Int. Cl. G16H 40/67; G16H 30/20; G16H 70/20

(52) U.S. Cl. CPC G16H 40/67; G16H 30/20; G16H 70/20

(57) ABSTRACT

Computer-implemented methods and facilities for providing a set of control commands for remote control of a medical imaging system arranged in a medical facility are based on providing a database with at least one set of control commands stored in the database for controlling medical imaging systems when performing medical imaging procedures. Furthermore, a request is acquired by a remote access facility from the medical facility for the performance of a medical imaging procedure with the medical imaging system, wherein said request includes target procedure information describing the imaging procedure to be performed. A set of control commands is provided to the medical facility based on a querying of the database using the target procedure information by the remote access facility.

### CROSS-REFERENCE TO RELATED APPLICATION(S)

The present application claims priority under 35 U.S.C. § 119 to German Patent Application No. 10 2022 208 589.2, filed Aug. 18, 2022, the entire contents of which are incorporated herein by reference.

### FIELD

One or more example embodiments of the present invention relate to facilities and methods for providing sets of control commands for controlling a medical imaging system and in particular an imaging modality. In particular, one or more example embodiments of the present invention relate to systems and methods for remote control of a medical imaging system and in particular an imaging modality by providing sets of control commands.

### BACKGROUND

Modern medical imaging processes or imaging procedures require well-established technical know-how on the part of the operating staff. Imaging procedures are often based on technically complex imaging methods using advanced medical imaging systems, such as, for example, computed tomography systems or magnetic resonance imaging systems requiring deep understanding of the mode of operation and the underlying imaging processes for adequate operation.

The performance of an imaging procedure is the responsibility of experts in medical technology, such as, for example radiologists or medical technologists who often have to be present at, or at least in the vicinity of, the imaging system during the entire imaging procedure.

However, healthcare providers are increasingly faced with non-availability of experienced staff. In particular, remote or rural locations suffer from a shortage of well-educated and trained staff for performing medical imaging methods on site. This results in waiting times or in particular the need for patients to travel and generally in inadequate health care.

To solve such problems, EP 3 799 075 A1 proposes that imaging procedures be performed at least partially remotely. For this purpose, technical experts are brought together at a central location where they can connect remotely to imaging systems in order to perform a requested imaging procedure remotely. To assign such appropriate experts to a requested imaging procedure, EP 3 799 075 A1 in particular proposes a scheduling function that manages the technical experts at the central location.

In practice, it has been identified that even such approaches are only able to alleviate, but cannot eliminate, the basic problem of a shortage of technical experts for performing medical imaging procedures. When demand is high, even such central facilities can experience significant waiting times for processing. A further problem is that the technical experts in such a “remote access facility” are often already busy with routine imaging procedures and thus are able to devote correspondingly less time to more complex imaging procedures.

### SUMMARY

It is therefore an object of the present invention to provide alternative means, mechanisms, facilities, devices and/or methods that ensure on-demand, rapid and resource-efficient processing of medical imaging procedures to be performed with medical imaging systems.

This and further objects are achieved by a method, a device, a facility, a (non-transitory) computer program product or a (non-transitory) computer-readable storage medium according to the main claim and the independent claims. Advantageous developments are disclosed in the dependent claims.

The following describes how a solution to the object is achieved according to one or more example embodiments of the present invention with respect to both the claimed facilities and with respect to claimed methods. Features, advantages or alternative embodiments mentioned herein are equally applicable to the other claimed subject matter and vice versa. In other words, the substantive claims (which are, for example, directed at a facility) can also be developed with the features described or claimed in connection with a method. Herein, the corresponding function features of the method are embodied by corresponding substantive modules.

According to one aspect, a computer-implemented method for providing a set of control commands for controlling a medical imaging system is provided. The medical imaging system is arranged in a medical facility. The method comprises a plurality of steps. A first step is directed at providing a database with at least one set of control commands stored in the database for controlling medical imaging systems when performing a medical imaging procedure. A further step is directed at acquiring a request from the medical facility in a remote access facility, wherein the request is directed at performing a medical imaging procedure with the medical imaging system and the request includes target procedure information, wherein the target procedure information identifies the imaging procedure to be performed. A further step is directed at the provision of a set of control commands (suitable for the imaging procedure to be performed) from the remote access facility to the medical facility, and, to be precise, based on querying the database using the target procedure information by the remote access facility.

The medical imaging system can generally be embodied to generate medical image data. Medical image data can generally be image data of a body part of a patient. Accordingly, medical imaging systems are embodied to map body parts of patients. In particular, medical imaging systems can implement radiological imaging methods. Medical imaging systems can include one or more imaging modalities, such as, for example, computed tomography devices, magnetic resonance imaging devices, X-ray devices or ultrasound devices and the like. The imaging modalities can be controlled by suitable control commands for performing an imaging procedure.

Furthermore, the medical imaging system can comprise further components that can be controlled by control commands for the performance of an imaging procedure, such as, for example, a patient support apparatus, an injection apparatus for administering medical agents to a patient during an imaging procedure, or an archiving apparatus for image data generated with the imaging modality.

The medical facility can, for example, be a hospital or a hospital group with a plurality of hospitals. Furthermore, the medical facility can be a section or a department within a hospital or hospital group, such as, for example, a radiology department. Furthermore, the medical facility can be a practice such as, for example, a radiology practice.

A medical facility can be arranged at one or more locations. A medical facility can comprise one or more medical imaging systems, which can in particular be arranged at different locations. A medical facility can comprise an internal medical information system for data exchange or generally for communication within the medical facility. The medical information system can comprise a communication interface for communication with facilities outside the medical facility.

The database is embodied to store a plurality of sets of control commands for controlling medical imaging systems. The database can comprise one or more storage facilities. The database can in particular comprise a server system. Furthermore, the database can be embodied as what is known as cloud storage.

The database can be embodied as a central database in the sense that it stores sets of control commands for a plurality of different imaging systems and/or a plurality of different medical facilities.

A set of control commands is generally suitable to be input into a medical imaging system and for being executed or implemented in the medical imaging system. A set of control commands can comprise one or more individual control commands. A set of control commands can be suitable for actuating one or more components of a medical imaging system. In particular, the set of control commands can be embodied to actuate a medical imaging modality. For this purpose, the set of control commands can, for example, include one or more parameters for setting the respective imaging modality, such as, for example, one or more magnetic resonance imaging sequences for a magnetic resonance imaging device. In addition, a set of control commands can comprise one or more parameters for setting, in particular simultaneously setting, a patient support apparatus or a contrast medium injection apparatus.

A set of control commands can be embodied such that, when the set of control commands is input into the medical imaging system, a corresponding medical imaging procedure is executed directly. Additionally or alternatively, the set of control commands can comprise one or more instructions for an operator, the execution of which by the operator leads to the performance of a corresponding imaging procedure. The instructions can be output as audio-based and/or visual instructions, for example by a medical imaging system.

An imaging procedure is directed at the generation of medical image data or a medical image data set by mapping a body part of a patient by an imaging modality. In addition to the actual imaging, the imaging procedure can include various preparatory, concomitant and/or subsequent procedure steps, such as, for example, supporting the patient on a patient support apparatus, contrast medium administration or processing of the generated image data for visualization. Individual procedure steps or all of these procedure steps can be addressed in a set of control commands.

The request can in particular be understood to be a request from the medical facility for assistance with an imaging procedure to be performed. Herein, the request includes target procedure information describing or identifying the imaging procedure to be performed. Moreover, the target procedure information can identify a patient on whom the imaging procedure is to be performed. Furthermore, the request can include a time stamp for the performance of the imaging procedure, for example a time window or deadline.

The request can, for example, be acquired in a remote access facility. In particular, the request can be received in the remote access facility. The request can be transmitted from the medical facility to the remote access facility.

The remote access facility can generally be embodied to receive or acquire and process requests from medical facilities, to host and/or manage the database and/or to provide one or more sets of control commands for the performance of a medical imaging procedure to be performed according to a request and in particular to transmit them to the (requesting) medical facility.

The remote access facility can in particular be arranged outside the medical facility or facilities. In alternative embodiments, the remote access facility can also be partially arranged within the medical facility. The remote access facility can include a server system and, in particular, a cloud server system. The remote access facility can be connected to one or more medical facilities. For this purpose, the remote access facility can comprise a communication interface embodied to establish data communication with one or more medical facilities.

To provide the set of control commands, the remote control facility can in particular be embodied to query the database based on the target procedure information for one or more suitable sets of control commands. In particular, this can include a query as to whether, based on the target procedure information, a set of control commands stored in the database is suitable for performing the imaging procedure to be performed.

Herein, “suitable” can in particular mean that the provided set of control commands is embodied such that, when the provided set of control commands is executed by the medical imaging system, the imaging procedure to be performed is partially or completely performed by the medical imaging system and/or that the performance of the imaging procedure to be performed is at least partially assisted by the medical imaging system (for example by instructions to operators).

Thus, the provision of the set of control commands can include determining the set of control commands based on querying the database using the target procedure information. The provision of the set of control commands can furthermore include transmitting the set of control commands from the remote access facility to the medical facility.

Providing a database with control commands and querying the database can enable requests for controlling imaging systems to be processed without necessarily involving an operator. If existing control commands can be assigned to a requested imaging procedure, they can be automatically disseminated. This can relieve the workload on operators in the medical facility and on operators in the remote access facility. Furthermore, resorting to stored command datasets enables requests to be processed more quickly. Ultimately, therefore, a more efficient and resource-saving method for (remote) control of medical imaging systems is provided.

According to one aspect, the method furthermore includes transmitting the provided set of control commands from the remote access facility to the medical facility and/or receiving the transmitted set of control commands from the medical facility and/or inputting the received set of control commands into the medical imaging system and/or controlling the medical imaging system based on the received set of control commands.

According to one aspect, the method furthermore includes the step of adapting the provided set of control commands based on the target procedure information, wherein the adapted set of control commands is provided in the step of providing.

Adapting the control command dataset enables the control command dataset to be better adapted to the imaging procedure to be performed. For example, this enables imaging parameters to be adapted to changed key data, such as, for example, those relating to the patient (height, weight, etc.). The adaptation step enables better outcomes to be achieved when the imaging procedure is performed locally.

According to some exemplary embodiments, adaptation can take place by inputting the provided set of control commands and the target procedure information into a trained function, wherein the trained function is embodied to adapt sets of control commands based on target procedure information about an imaging procedure to be performed to the imaging procedure to be performed.

According to one aspect, the database is arranged in the remote access facility. In particular, the database can be arranged outside the medical facility. In particular, the database can be arranged at a location that is different from a location of the medical facility.

Arrangement in the remote access facility enables the database to hold sets of control commands for different medical facilities and imaging systems. This increases the flexibility of the method.

According to one aspect, the step of providing includes:
querying, based on the target procedure information, whether the imaging procedure to be performed can be performed with a set of control commands stored in the database,
if yes: providing the stored set of control commands as a set of control commands to the medical facility, and
if no: providing the set of control commands by:
   assigning a remote operator at the remote access facility for remote control of the imaging procedure to be performed by the remote operator from the remote access facility.
   acquiring a set of remote control commands input into the remote access facility by the remote operator for the performance of the target imaging procedure in the remote access facility,
   providing the set of remote control commands as a set of control commands to the medical facility.

The querying can include checking the sets of control commands present in the database as to whether the sets of control commands are suitable for the imaging procedure to be performed. This can take place based on the target procedure information.

A remote operator can in particular be a physician, a medical technologist or another expert in the operation of imaging systems. The remote operator can in particular be allocated organizationally to the remote access facility.

The remote operator can, for example, be assigned based on the target procedure information. A remote operator can, for example, have a profile or be allocated to a profile that identifies the remote operator in respect of imaging procedures to be performed. Accordingly, the remote operator can be assigned based on the target procedure information and/or the profile and in particular based on a comparison of the target procedure information with the profile. Furthermore, the assignment of the remote operator can include a selection from a plurality of available remote operators based on the target procedure information and/or the profiles of the remote operators.

The set of remote control commands can in particular be constructed like the stored sets of control commands. Accordingly, the set of remote control commands is likewise suitable to be input into a medical imaging system and executed or implemented in the medical imaging system. The set of remote control commands can also comprise one or more individual control commands. In particular, the set of remote control commands can be embodied to actuate the medical imaging system such that the medical imaging system executes the imaging procedure to be performed.

The set of remote control commands can be input into the remote access facility by the remote operator, for example via a corresponding remote access workstation of the remote access facility, and acquired there. The acquisition and provision can take place continuously while the individual control commands are input. Therefore, it is not necessary to wait until the input of the set of remote control commands by the remote operator has been fully completed.

The optional assignment of a remote operator enables a set of control commands to be transmitted to the medical facility even if the database does not contain a suitable set of control commands. This can efficiently assist the medical facility when performing imaging procedures.

According to one aspect, the database includes at least one data element that links the at least one stored set of control commands to procedure information describing or identifying an imaging procedure that can be performed with the set of control commands, wherein the querying of the database takes place based on a comparison of the target procedure information with the procedure information.

Herein, the procedure information can be constructed in the same way as the target procedure information. Comparing the procedure information enables reliable identification of suitable sets of control commands.

According to one aspect, the set of remote control commands includes at least one visual and/or audio-based instruction to an operator in the medical facility for the performance of the imaging procedure to be performed (or the set of remote control commands is embodied to induce in the medical facility an output of at least one visual and/or audio-based instruction to an operator for the performance of the imaging procedure to be performed). The method furthermore includes the steps:
logging at least one operator action performed by the local operator based on the set of remote control commands on the medical imaging system,
determining at least one further set of control commands based on the logged operator actions and
storing the at least one further set of control commands in the database.

The logging of the operator action can, for example, include recording the operator action in the medical facility and/or transmitting the recorded operator actions to the remote access facility.

Logging operator actions and translating them into sets of control commands enables the scope of the database to be expanded so that a broader spectrum of requests can be processed in future without remote operators. This can successively improve the efficiency of the method.

According to one aspect, storing the at least one further set of control commands includes the creation of a further data element based on the target procedure information and the further set of control commands and adding the further data element to the database.

According to one aspect, the method furthermore includes ascertaining a quality measure of the operator action performed, wherein the steps of determining and storing the at least one further set of control commands take place based on the step of ascertaining and in particular are only performed if the quality measure meets a prespecified quality criterion.

For example, ascertaining a quality measure can include determining the time taken for the operator action, an assessment of corrections within the operator action and/or the acquisition of a number of individual steps within the operator action. Furthermore, ascertaining a quality measure can include an analysis of a medical image data set generated by the operator action. Herein, it is in particular possible to determine whether the mapped body part and the components thereof are completely mapped and have good resolution. Furthermore, values, such as contrast, sharpness, image homogeneity, image noise etc., can be read from the image data set on the basis of which the quality of the image data set, and thus a quality measure of the operator action, is determined.

The quality measure can, for example, be a numerical value, such as, for example, an integer, that specifies the quality of the operator action to be performed. In particular, the quality measure can specify the quality of the imaging procedure on a scale, for example from 1 (low) to 10 (high). The quality criterion can, for example, be a threshold value that the quality measure must exceed to meet the criterion.

The quality check enables it to be ensured that only operator actions of a certain quality standard are converted into control command sets and used further.

According to one aspect, the method furthermore includes the steps:
determining a complexity level of the imaging procedure to be performed based on the target procedure information,
comparing the complexity level with a prespecified threshold value,
wherein, if the complexity level is above the prespecified threshold value, the set of control commands is provided as a set of remote control commands and not as a stored set of control commands.

The complexity level can, for example, be a numerical value, such as, for example, an integer, that specifies the complexity or difficulty of the imaging procedure to be performed. In particular, the complexity level can specify the complexity of the imaging procedure on a scale, for example from 1 (low) to 10 (high). Accordingly, the complexity level for the imaging procedure to be performed can be selected from a plurality of predetermined values.

Taking account of the complexity of the imaging procedure to be performed enables a selective decision to be made as to whether the provision of stored sets of control commands is sufficient or whether a remote operator needs to be connected. This enables better assistance for imaging procedures that require more assistance.

According to one aspect, the method furthermore includes the steps:
receiving a medical image data set as the outcome of the imaging procedure to be performed,
adapting the provided set of control commands based on the image data set, in particular including adaptation of at least one set of control commands stored in the database and/or adding a further set of control commands to the database based on the adapted provided set of control commands.

The reception of the image data set can in particular include transmitting the image data set from the medical facility to the remote access facility and receiving the image data set in the remote access facility.

The provided set of control commands can, for example, be adapted by applying a trained function to the image data set, wherein the trained function is embodied, based on an image data set, to adapt a set of control commands (and in particular imaging parameters contained therein) used in the generation of the image data set. Optionally, the trained function can furthermore be embodied to additionally take account of the target procedure information during the adaptation.

Furthermore, the quality of the medical image data set, can be determined, optionally further taking into account the target procedure information. The set of control commands can then be adapted based on the determined quality.

The retroactive adaptation of the set of control commands based on the generated image data—and in particular based on the quality of the image data—enables the quality of the sets of control commands to be systematically improved. Thus, a more efficient and resource-saving method for controlling medical imaging systems is provided.

According to one aspect, the imaging procedure to be performed includes a plurality of different procedure steps, wherein a set of control commands is provided for each procedure step.

According to one aspect, the imaging procedure to be performed comprises a plurality of different procedure steps. The method furthermore includes the steps:
determining a complexity level for each procedure step based on the target procedure information, and
for each procedure step: ascertaining whether the complexity level exceeds a prespecified threshold value, wherein the step of providing for the respective procedure step only takes place if the complexity level of the respective method step is above the prespecified threshold value.

Differentiation according to procedure steps in the complexity assessment makes it possible to ensure that sets of control commands are only provided for procedure steps for which the medical facility actually requires assistance. On the other hand, trivial procedure steps can be executed by operators of the medical facility without the provision of sets of control commands. This provides a more efficient and resource-saving method for controlling medical imaging systems.

According to one aspect, the method furthermore includes the steps:
receiving an operator action of an operator in the medical facility when performing an imaging procedure and procedure information describing the imaging procedure,
ascertaining a comparison set of control commands based on querying the database using the procedure information,
determining an efficiency value of the operator action based on a comparison between the operator action and the comparison set of control commands and
providing the efficiency value.

The efficiency value can, for example, be a numerical value, such as, for example, an integer, that specifies the efficiency of the operator action performed (in particular compared to the comparison set of control commands). In particular, the complexity level can specify the efficiency of the operator action on a scale, for example from 1 (low) to 10 (high). In particular, a high efficiency value can be determined if, based on the comparison, a high level of conformity with the comparison set of control commands was identified. The ascertainment of the efficiency value can take account of operator actions performed based on sets of control commands (instructions) provided by the remote access facility or of those performed independently thereof by operators of the medical facility on the medical imaging systems.

The comparison set of control commands can be ascertained by querying the database based on the procedure information. Herein, the procedure information can have the same form as the target procedure information and the database can be queried in the same way as explained in connection with the target procedure information.

Ascertaining an efficiency value enables a medical facility to be provided with a further service based on the database. Thus, it is not only possible for sets of control commands to be provided, the database also enables systematic and objective checking of operator actions. Thus, it is possible to identify a potential for optimization within the medical facility and this likewise contributes to a more efficient and resource-saving method for controlling medical imaging systems.

According to one aspect, the database is arranged in the medical facility and stores sets of control commands specific to the medical facility.

Although the database is arranged in the medical facility, the database can be controlled or managed from the remote access facility. This enables the method steps relating to the database to be implemented by the remote access facility. At the same time, sets of control commands can be better adapted to the medical facility and it is possible to ensure that sensitive information does not leave the medical facility, thus improving data security. At the same time, data flows are reduced.

In particular, the remote access facility is embodied such or the method is implemented such that the target procedure information is only exchanged between the medical facility and the database arranged within the medical facility—i.e., this also does not leave the medical facility. In particular, the database can be implemented as what is known as an edge device in the medical facility network. In particular, the database and the functions/steps of acquiring the target procedure information and/or of providing the set of control commands can at least partially be executed “on edge” in the medical facility network.

According to some exemplary embodiments, the remote access facility is in data communication with a plurality of different medical facilities. Herein, a plurality of these medical facilities can comprise a database arranged in the respective medical facility. At the same time or in addition, the remote access facility can comprise a central database.

According to one aspect, the target procedure information includes one or more of the following elements:
specifying a body part of a patient to be mapped with the imaging procedure to be performed,
specifying a diagnostic context of the imaging procedure,
specifying different individual procedure steps to be performed within the imaging procedure,
specifying the medical imaging system to be used within the imaging procedure and/or
specifying a complexity level of the imaging procedure and/or individual procedure steps of the imaging procedure.

The diagnostic context can in particular refer to the diagnostic task to be performed for the patient by the medical facility and/or a suspected diagnosis to be confirmed or disproved.

The target procedure information enables the imaging procedure to be performed to be well characterized with clear data streams—this, on the one hand, enables a targeted selection of sets of control commands and, on the other hand, ensures data security.

According to some exemplary embodiments, the target procedure information is anonymized and/or pseudonymized to suppress the dissemination of confidential patient data.

According to one aspect, the medical imaging system includes one or more of the following elements:
a medical imaging device, in particular an X-ray device, a computed tomography device, a magnetic resonance imaging device, a positron emission tomography device, an ultrasound device or a radiotherapy device,
a radiology information system,
a picture archiving and communication system,
a treatment scheduling system,
a patient positioning system and/or
a injection facility for substance administration to a patient, in particular a contrast medium injector.

The aforementioned components of the medical facility allow the performance of complex imaging procedures, in particular those requiring interaction between a plurality of different subsystems.

According to one aspect, the provision of a set of control commands that can be used for the imaging procedure to be performed includes:
providing a trained function embodied, based on target procedure information, to generate a set of control commands for the performance of an imaging procedure.

In synopsis, therefore, according to some embodiments, trained functions can be used for:
a) the provision of sets of control commands,
b) the adaptation of sets of control commands (before their use/dissemination) in the step of providing based on the target procedure information and/or
c) the subsequent adaptation of sets of control commands based on image data generated with the respective set of control commands.

A trained function generally maps input data to output data. The output data can in particular furthermore be dependent on one or more parameters of the trained function. The one or more parameters of the trained function can be determined and/or adapted by training. In general, a trainable function, i.e., a function with parameters that have not yet been adapted, is also referred to as a trained function.

Other terms for trained function are trained mapping rule, mapping rule with trained parameters, function with trained parameters, algorithm based on artificial intelligence, machine learning algorithm. One example of a trained function is an artificial neural network. The term “neural net” can also be used instead of the term “neural network”. A neural network is basically constructed like a biological neural network such as, for example, a human brain. In particular, an artificial neural network includes an input layer and an output layer. It can furthermore include a plurality of layers between the input layer and the output layer. Each layer includes at least one, preferably multiple, nodes. Each node can be understood as a biological processing unit, for example a neuron. In other words, each neuron corresponds to an operation applied to input data. Nodes in one layer can be connected to nodes in another layer by edges or connections, in particular directed edges or connections. These edges or connections define the data flow between the nodes in the network. The edges or connections are associated with a parameter often referred to as a “weight” or “edge weight”. This parameter can regulate the importance of the output of a first node for the input of a second node, wherein the first node and the second node are connected by an edge.

In particular, a neural network can be trained. In particular, training of a neural network is performed based on training input data and associated training output data according to a “supervised” learning technique, wherein the known training input data is input into the neural network and the output data generated by the network is compared with the associated training output data. The artificial neural network learns and adapts the edge weights for the individual nodes independently as long as the output data of the last network layer does not sufficiently match the training output data.

In particular, a trained function can also be a deep artificial neural network or deep neural network. According to some implementations, the trained function comprises a neural network and in particular a convolutional neural network. In particular, the convolutional neural network can be embodied as a deep convolutional neural network. Herein, the neural network comprises one or more convolutional layers and one or more deconvolutional layers. In particular, the neural network can include a pooling layer. The use of convolutional layers and/or deconvolutional layers enables a neural network to be used efficiently for deriving a parameter set since, despite the many connections between node layers, only a few edge weights (namely the edge weight corresponding to the values of the convolutional kernel) need to be determined. Hence, it is also possible to improve the accuracy of the neural network with the same amount of training data. In particular, it has been found that convolutional neural networks are able to efficiently process volume data as input data.

For example, a dataset for training the trained function can include training input data and training output data. The determination and/or adaptation of one or more parameters of the trained function can in particular be based on a pair consisting of training input data and associated training output data, wherein the trained function is applied to the training input data to generate training mapping data. In particular, the determination and/or adaptation can be based on a comparison of the training mapping data and the training output data. The following training input data and training output data can be provided and the following training mapping data can be generated for the different applications a)-c):

| | Training input data | Training output data |
|---|---|---|
| a ) | Target procedure | (Verified) set of |
| | information | control commands |
| b) | Target procedure | (Verified) set of |
| | information + set | control commands |
| | of control commands | |
| | to be adapted | |
| c) | Image data + set | (Verified) set of |
| | of control commands | control commands |
| | to be adapted + | |
| | (optional) target | |
| | procedure | |
| | information | |

Verified sets of control commands and the associated target procedure information can be easily provided by existing inputs or data elements in the database. Alternatively or additionally, datasets for training can be provided based on remote control commands provided by a remote operator. Image data for application c) is easy to provide based on the associated image data, i.e., based on the image data generated with the respective sets of control commands.

According to one aspect, a method for providing a trained function is provided, which is embodied to provide a (possibly adapted) set of control commands with which a medical imaging system can be actuated. The method comprises a plurality of steps. A first step is directed at providing training input data, wherein the training input data comprises target procedure information (and optionally image data and/or a set of control commands to be adapted). A further step is directed at providing training output data, wherein the training output data includes a verified set of control commands. A further step is directed at generating a set of control commands by applying the trained function to the training input data, wherein the set of control commands is suitable for inducing a medical imaging procedure when input into a medical imaging system. A further step is directed at comparing the set of control commands with the training output data. A further step is directed at adapting the trained function based on the comparison.

According to a further aspect, a training system for providing a trained function is provided, which is embodied to execute one or more method steps of the aforementioned method for providing a trained function.

The use of a trained function enables the set of control commands to be efficiently generated automatically. Compared to rule-based generation, the use of a trained function has the advantage that the trained function is enabled by the training to create dynamic adaptation to different circumstances.

According to one aspect, a (remote access) facility for providing a set of control commands for controlling a medical imaging system in a medical facility is provided, wherein the facility comprises:
a communication interface in data communication with the medical facility,
a database with at least one set of control commands stored in the database for controlling the medical imaging system when performing a medical imaging procedure and
a computing unit embodied to:
   acquire a request for the performance of a medical imaging procedure with the medical imaging system from the medical facility via the communication interface, wherein the request includes target procedure information, wherein the target procedure information identifies the imaging procedure to be performed and
   provide a set of control commands that can be used for the imaging procedure to be performed based on querying the database using the target procedure information to the medical facility via the communication interface.

For example, the computing unit can include a database management module embodied, based on the target procedure information, to query the database for suitable sets of control commands. For example, the computing unit can include a providing module embodied to provide the medical facility with a suitable set of control commands via the communication interface.

The computing unit can be embodied as a server system. The computing unit can comprise a cluster or a group of computing facilities and data storage devices. The computing unit can comprise a user interface for a remote operator. The computing unit can be in data communication with the medical facility via a network such as, for example, the internet. The computing unit can be in data communication with a plurality of different medical facilities. The computing unit can be embodied within or outside the medical facilities.

The communication interface can generally be embodied for data exchange between the computing unit and further components. The communication interface can be implemented in the form of one or more individual data interfaces, which can comprise a hardware and/or software interface. The communication interface can furthermore comprise an interface of a communication network, wherein the communication network can comprise a wide area network (WAN) or the internet.

The advantages of the proposed facility substantially correspond to the advantages of the proposed method. Features, advantages or alternative embodiments can likewise be transferred to the other claimed subject matter and vice versa.

According to one aspect, a computer program product with a computer program is provided, which can be loaded directly into a memory of a facility, with program sections for executing all steps of the method for providing a set of control commands or for providing a trained function according to one of the aspects described herein when the program sections are executed by the facility.

According to one aspect, a computer-readable storage medium is provided on which program sections that can be read and executed by a facility are stored in order to execute all steps of the method for providing a set of control commands or for providing a trained function according to one of the aspects described herein when the program sections are executed by the facility.

Herein, the computer program products can include software with a source code, which still has to be compiled and linked or only has to be interpreted, or an executable software code that only needs to be loaded into the processing unit for execution. The computer program products enable the method to be executed quickly, identically repeatedly and robustly. The computer program products are configured such that they can execute the method steps according to embodiments of the present invention via the computing unit. Herein, the computing unit must in each case fulfil the requisite requirements such as, for example, having an appropriate random-access memory, an appropriate processor, an appropriate graphics card or an appropriate logic unit so that the respective method steps can be executed efficiently.

The computer program products are, for example, stored on a computer-readable storage medium or held on a network or server from where they can be loaded into the processor of the respective computing unit—this can be directly connected to the computing unit or embodied as part of the computing unit. Furthermore, control information of the computer program products can be stored on a computer-readable storage medium. The control information of the computer-readable storage medium can be embodied to perform a method according to embodiments of the present invention when the data medium is used in a computing unit. Examples of computer-readable storage media are DVDs, magnetic tapes or USB sticks on which electronically readable control information, in particular software, is stored. When this control information is read from the data medium and stored in a computing unit, all the embodiments of the above-described methods can be performed. Thus, embodiments of the present invention can also be based on said (non-transitory) computer-readable medium and/or said (non-transitory) computer-readable storage medium.

The advantages of the proposed computer program products or the associated computer-readable media substantially correspond to the advantages of the proposed method.

### BRIEF DESCRIPTION OF THE DRAWINGS

Further special features and advantages of the present invention will become apparent from the following explanations of exemplary embodiments based on schematic drawings. Modifications mentioned in this context can in each case be combined with one another in order to form new embodiments. In different figures, the same reference characters are used for the same features.

In the drawings:

FIG. 1 shows a schematic representation of a system for providing a set of control commands for the performance of a medical imaging procedure according to one embodiment,

FIG. 2 shows a schematic representation of a system for providing a set of control commands for the performance of a medical imaging procedure according to a further embodiment,

FIG. 3 shows a flowchart of a method for providing a set of control commands for the performance of a medical imaging procedure according to one embodiment,

FIG. 4 shows a flowchart of optional substeps of a method for providing a set of control commands for the performance of a medical imaging procedure according to one embodiment,

FIG. 5 shows a flowchart of optional substeps of a method for providing a set of control commands for the performance of a medical imaging procedure according to one embodiment,

FIG. 6 shows a flowchart of optional substeps of a method for providing a set of control commands for the performance of a medical imaging procedure according to one embodiment,

FIG. 7 shows a flowchart of optional substeps of a method for providing a set of control commands for the performance of a medical imaging procedure according to one embodiment and

FIG. 8 shows a flowchart of a method for providing an efficiency assessment when performing a medical imaging procedure according to one embodiment.

### DETAILED DESCRIPTION

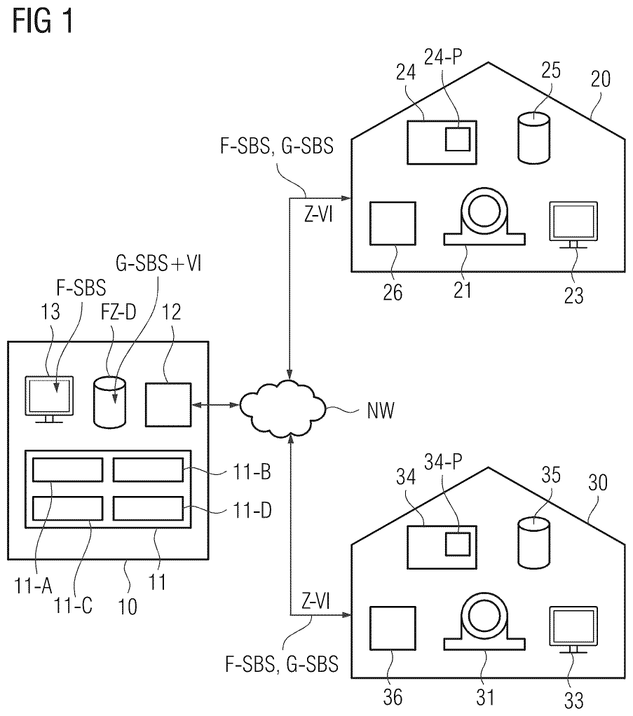

FIG. 1 shows a system 1 for providing a set of control commands F-SBS, G-SBS for controlling a medical imaging system 21, 31 for performing a medical imaging procedure according to one embodiment. The system 1 is embodied to perform methods for providing a set of control commands F-SBS, G-SBS according to one or more embodiments described herein. The system 1 includes components and/or facilities that can be arranged in a distributed environment. Therefore, individual system components and facilities can be located at different locations.

The system 1 includes a remote access facility 10 and at least one medical facility 20, 30. The medical facilities 20, 30 can, for example, be hospitals, radiology departments, radiology practices, etc. The medical facilities 20, 30 in each case comprise one or more medical imaging systems 21, 31.

The remote access facility 10 can (at least partially) be arranged at a location different from the locations of the medical facilities 20, 30, preferably a central location. However, alternatively, the remote access facility 10 can also at least partially be arranged in a medical facility 20, 30. The remote access facility 10 is embodied to provide the medical imaging systems 21, 31 with sets of control commands G-SBS, F-SBS and thereby at least partially to control the imaging systems 21, 31 remotely.

The medical imaging systems 21, 31 of the medical facilities 20, 30 can in particular include imaging modalities such as computed tomography devices, magnetic resonance imaging devices, X-ray devices, ultrasound devices, angiography devices, etc. However, other types of imaging modalities are likewise possible. In addition, medical imaging systems 21, 31 can include peripheral devices such as patient support apparatuses, injection apparatuses, for example for contrast medium administration, picture archiving and communication systems (PACS). The imaging modalities are configured to generate medical image data as the outcome of a medical imaging procedure.

The medical facilities 20, 30 can furthermore in each case include operator workstations 23, 33 for (local) operators, which in turn can include an input unit and an output unit Furthermore, the operator workstations 23, 33 can be embodied to visualize image data generated with the imaging systems 21, 31 in graphical form as part of an imaging procedure. The workstations 23, 32 can be embodied to depict in graphical form a graphical user interface for scheduling an imaging procedure or a corresponding protocol. In addition, the operator workstations 22, 32 can be embodied to visualize a chat window that enables written communication and/or video communication with a remote operator located in the remote access facility 10. The operator workstations 22, 32 can furthermore be embodied to output audio chat signals. The operator workstations 22, 32 can be embodied to receive user input relating to an imaging procedure. The user input can include an operator action relating to the performance of an imaging procedure (i.e., local input of control commands for controlling the imaging systems 21, 31 by a local operator) and/or input relating to the scheduling of the imaging procedure. The operator workstations 22, 32 can include an LCD, plasma or OLED screen or another type of display and an input facility, such as a touch-sensitive screen, keyboard, mouse, joystick etc. The operator workstations 22, 32 can be arranged on the medical imaging systems 21, 31.

The medical facilities 20 and 30 can in each case include a computing unit 24 and 34. The computing units 24, 34 can be embodied to manage electronic files for patients (patient records or electronic health records) stored, for example, in an electronic patient register 25, 35, of the respective medical facility 20, 30. Electronic health records can include patient-related data that, for example, identifies the patient or indicates the patient's medical condition. Furthermore, electronic health records can include referral documents, electronic preliminary reports, previous or current suspected diagnoses, recommended treatment or a specification for a scheduled medical imaging procedure or existing image data or the like.

The computing units 24, 34 can in particular in each case include a scheduling module 24-P, 34-P embodied to schedule and/or coordinate medical imaging procedures within the respective medical facility 20, 30. In particular, such scheduling modules 24-P, 34-P can be embodied to create target procedure information Z-VI for an imaging procedure to be performed, for example based on the electronic patient records, wherein the target procedure information Z-VI identifies the imaging procedure to be performed. For example, the target procedure information Z-VI can specify which body part is to be mapped with which type of medical imaging system. Furthermore, the target procedure information Z-VI can specify the urgency and/or complexity level of the imaging procedure to be performed. For this purpose, the scheduling module can, for example, be embodied to select a complexity level for the respective imaging procedure from a plurality of predetermined complexity levels and assign it to the imaging procedure. Furthermore, the target procedure information Z-VI can specify a plurality of individual procedure steps to be performed during the imaging procedure, such as, for example, different individual scans with different imaging parameters.

The scheduling modules 24-P, 34-P can furthermore be embodied to provide an electronic schedule that specifies when, or in which window, an imaging procedure is to be performed with which imaging system in the respective local facility 20, 30. Furthermore, the scheduling modules 24-P, 34-P can be embodied to formulate an electronic request REQ to the remote access facility 10 in order to have the imaging procedure to be performed assisted by the provision of sets of control commands F-SBS, G-SBS. The request REQ can include the time window and/or the target procedure information Z-VI.

The computing units 24, 34 of the medical facilities 20, 30 can be implemented as servers including a microcontroller or an integrated circuit. The computing units 24, 34 can include hardware elements and/or software elements, such as, for example, a microprocessor or an FPGA (field programmable gate array). According to some embodiments, the computing units 24, 34 are part of medical information systems of the respective medical facility 20, 30. For example, this system can be a hospital information system or a radiology information system.

The components of the medical facilities 20, 30 can be connected via one or more data interfaces (not shown) that ensure data exchange between the components of the local facilities 20, 30. The one or more data interfaces can comprise a hardware interface and/or software interface. The one or more data interfaces can comprise an interface of a communication network, wherein the communication network can comprise a local area network (LAN), for example an intranet. Accordingly, the one or more data interfaces can comprise a LAN interface or a wireless LAN interface (WLAN or Wi-Fi).

The medical facilities 20, 30 can furthermore in each case comprise a communication interface 26, 36 embodied for bidirectional data exchange with facilities outside the medical facility 20, 30 and in particular the remote access facility 10. Herein, the data exchange can take place via a network NW such as, for example, the internet. Accordingly, the communication interfaces 26, 36 can be embodied to use corresponding communication protocols, such as, for example, https or http protocol. The medical facilities 20, 30 can transmit requests REQ to the remote access facility 10 via the communication interfaces 26, 36 and in return receive, for example, sets of control commands F-SBS, G-SBS for local use, i.e., for actuating the imaging systems 21, 31.

The remote access facility 10 comprises a computing unit 11, a communication interface 12, at least one remote access workstation 13 and a (central) database FZ-D. The remote access facility 10 can in particular be embodied as remote from the medical facilities 20, 30. In particular, the remote access facility 10 can be understood as an external service center to which the one or more medical facilities 20, 30 are attached. Alternatively, the remote access facility 10 can also be implemented as an internal service center arranged in a medical facility 20, 30.

Via the communication interface 12, the remote access facility 10 is in data communication with the medical facilities 20, 30 via corresponding communication interfaces 26, 36 of the medical facilities 20, 30. Accordingly, the communication interface 12 is embodied for bidirectional data exchange with one or more medical facilities 20, 30. Herein, the data exchange can take place via the network NW. Accordingly, the communication interface 12 can be embodied to use corresponding communication protocols, such as, for example, https or http protocol. The remote access facility 10 can receive requests REQ from the medical facilities 20, 30 via the communication interface 12 and in return transmit, for example, sets of control commands F-SBS, G-SBS for local use, i.e., for actuating the imaging systems 21, 31.

The database FZ-D is embodied to store a plurality of control command datasets G-SBS. The stored sets of control commands G-SBS are suitable for actuating imaging systems 21, 31 for the performance of an imaging procedure. In particular, the sets of control commands G-SBS can be suitable for being directly input into and executed by a respective imaging system 21, 31. Furthermore, the sets of control commands G-SBS can be suitable for being first emulated or compiled for or applied to the respective imaging system 21, 31 in the medical facility for subsequent input into an imaging system 21, 31. Herein, a stored set of control commands G-SBS can be suitable for actuating an imaging procedure completely or for actuating only one or more individual procedure steps of an imaging procedure. The stored sets of control commands G-SBS can in each case be specific to an imaging system 21, 31, for example a sort or type or brand etc. Additionally or alternatively, the sets of control commands G-SBS can be specific to an imaging procedure to be performed. Additionally or alternatively, the sets of control commands G-SBS can be specific to a medical facility 20, 30.

For managing and querying stored sets of control commands G-SBS, the stored sets of control commands G-SBS in the database FZ-D can in each case be linked to procedure information VI describing the imaging procedure to be performed with the respective set of control commands G-SBS. In particular, the database FZ-D can comprise corresponding data elements that link stored sets of control commands G-SBS with the associated procedure information VI. Herein, the procedure information VI can in principle contain the same information as the target procedure information Z-VI.

The database FZ-D can be embodied as a centralized or decentralized memory facility. The database FZ-D can in particular be part of a server system. The database FZ-D can furthermore be embodied as what is known as a cloud memory.

The remote access workstation 13 in each case includes a user interface with an input unit and an output unit. The output unit can be embodied to visualize generated image data for a remote operator at the remote access workstation 13 in graphical form. The output unit can be embodied to display in graphical form a graphical user interface for scheduling and controlling an imaging procedure or a corresponding protocol. In addition, the output unit can be embodied to visualize a chat window that enables written communication and/or video communication with a local operator in the medical facilities 20, 30. The output unit can furthermore be embodied to output audio chat signals.

The input unit can be embodied to acquire user input from a remote operator and provide it, for example, in the form of one or more sets of control commands F-SBS (hereinafter also called remote sets of control commands F-SBS for differentiation from the stored sets of control commands G-SBS). The remote sets of control commands F-SBS can be suitable for being directly input into and executed by a respective imaging system 21, 31. Furthermore, the remote sets of control commands F-SBS can be suitable for being first emulated or compiled for or applied to the respective imaging system 21, 31 in the medical facility 20, 30 for subsequent input into an imaging system 21, 31. Herein, a set of remote control commands F-SBS can be suitable for actuating an imaging procedure completely or only partially, for example by addressing one or more individual procedure steps of an imaging procedure. The remote sets of control commands F-SBS can in each case be specific to an imaging system 21, 31. Additionally or alternatively, the remote sets of control commands F-SBS can be specific to an imaging procedure to be performed. Additionally or alternatively, the remote sets of control commands F-SBS can be specific to a medical facility 20, 30.

Additionally, the input unit can be embodied to enable written or audio-based input or video chat input for the remote operator for transmission to one of the local facilities 20, 30.

Accordingly, the remote access workstation 13 can include an LCD, plasma or OLED screen or another type of display. Additionally or alternatively, the remote access workstation 13 can include a touch-sensitive screen, keyboard, mouse or microphone and a speaker. For example, the remote access workstation 13 can include a desktop PC or laptop with one or more monitors.

The computing unit 11 is embodied, based on a request REQ transmitted by a medical facility 20, 30 or target procedure information Z-VI, to provide at least one set of control commands F-SBS, G-SBS to the local facility 20, 30. The computing unit 11 can comprise one or more processors. The processors can be implemented as a central processing unit (CPU), graphics processing unit (GPU), digital signal processor (DSP), an image processing processor, integrated (digital or analog) circuit or combinations of the aforementioned components. The computing unit 11 can be implemented as an individual component or comprise a plurality of components operating in parallel or in series. Alternatively, the computing unit 11 can comprise a real or virtual group of computers, such as, for example, a cluster or cloud. Depending on the embodiment, the computing unit 11 can be embodied as a local server or cloud server. The computing unit 11 is, for example, embodied by computer-readable instructions, by design and/or hardware such that it is able to execute one or more method steps according to embodiments of the present invention.

For the provision of the sets of control commands F-SBS, G-SBS, the computing unit 11 can comprise various modules 11-A, 11-B, 11-C and 11-D. Herein, the computing unit 11 is only subdivided into modules 11-A, 11-B, 11-C and 11-D for the purpose of a simpler explanation of the mode of operation of the computing unit 11 and should not be understood as being restrictive. The modules 11-A, 11-B, 11-C and 11-D or the functions thereof can also be combined in one unit. Herein, the modules 11-A, 11-B, 11-C and 11-D can in particular also be understood as computer program products or computer program sections which, when executed in the computing unit 11, implement one or more of the method steps described below.

Module 11-A can be understood as a process control module. Module 11-A can in particular be embodied, based on a request REQ and in particular on target procedure information Z-VI, to decide the way in which a set of control commands F-SBS, G-SBS is to be provided. For example, based on a complexity level contained in the target procedure information Z-VI or derived from the target procedure information Z-VI, it is possible to determine whether a remote operator needs to be involved or whether transmission of stored sets of control commands G-SBS is sufficient. Furthermore, module 11-A can be embodied to acquire input operator actions in a medical facility 20, 30 in order, after optional quality control, to convert them into further sets of control commands and store them in the database FZ-D. Furthermore, module 11-A can be embodied to store remote sets of control commands F-SBS provided by a remote operator—possibly after optional quality control—in the database FZ-D. Furthermore, module 11-A can be embodied to adapt stored sets of control commands G-SBS based on quality control. Quality control can, for example, take place based on the image data created during an imaging procedure. Additionally or alternatively, quality control can take place based on the time required for the imaging procedure or based on the corrections performed in the imaging procedure.

According to one aspect, module 11-A can furthermore be embodied to host and execute a trained function embodied to generate or adapt sets of control commands. Such a trained function can, for example, be trained based on stored sets of control commands G-SBS and associated procedure information VI.

Module 11-B can be understood as a database management module. Module 11-B can in particular be embodied to query the database FZ-D in order, for example, to check whether the database FZ-D contains a suitable control command dataset G-SBS for provision to a medical facility 20, 30. For this purpose, module 11-B can be embodied to compare target procedure information Z-VI with procedure information VI in the database FZ-D. If no matching stored set of control commands G-SBS can be found, module 11-B is furthermore embodied to report this back to the process control module 11-A. Furthermore, module 11-B can be embodied to add further sets of control commands to the database FZ-D.

Module 11-C can be understood as a remote control scheduling module. Module 11-C can in particular be embodied to assign a remote operator to a request REQ. In particular, module 11-C becomes active if the process control module 11-A determines that a remote operator needs to be involved, for example because no suitable stored set of control commands G-SBS can be found or because the imaging procedure to be performed has a complexity level requiring the involvement of a remote operator. Module 11-C can, for example, assign remote operators based on the availability of remote access workstations 12, the expertise and availability of remote operators and the time window specified in the request REQ.

Module 11-D can be understood as a providing module. Module 11-D can be embodied to transmit one or more sets of control commands G-SBS, F-SBS to the medical facility 20, 30.

The components of the remote access facility 10 can be connected via one or more data interfaces (not shown) that ensure data exchange between the components of the remote access facility 10. The one or more data interfaces can comprise a hardware interface and/or software interface. The one or more data interfaces can comprise an interface of a communication network, wherein the communication network can comprise a local area network (LAN), for example an intranet or a wide area network (WAN) or an internet. Accordingly, the one or more data interfaces can comprise a LAN interface or a wireless LAN interface (WLAN or Wi-Fi).

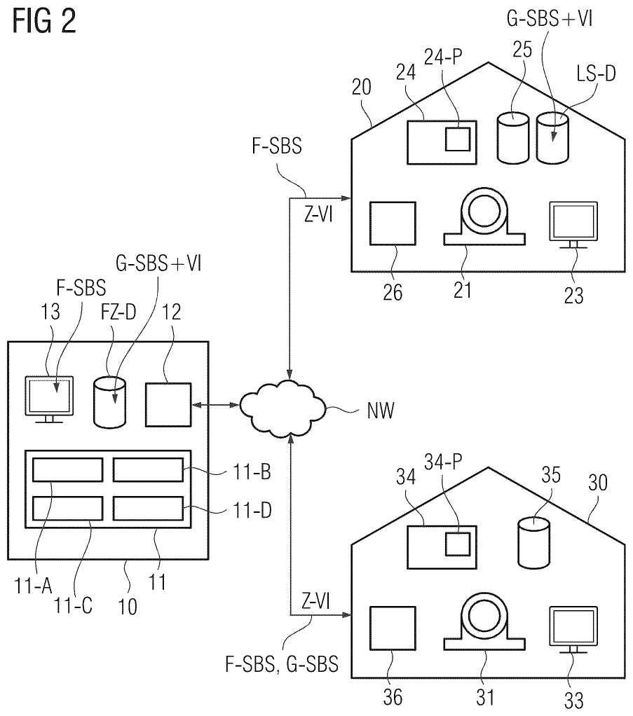

FIG. 2 shows a system 1 for providing a set of control commands F-SBS, G-SBS for controlling a medical imaging system 21, 31 for performing a medical imaging procedure according to one embodiment. The system 1 is embodied to perform methods for providing a set of control commands F-SBS, G-SBS according to one or more embodiments described herein.

The system 1 shown in FIG. 2 differs from the system 1 in FIG. 1 in that at least one medical facility 20 comprises a local database LS-D in which sets of control commands G-SBS specific to the respective facility are stored. Further components correspond to the components explained in connection with FIG. 1. In particular, the same reference characters denote identical or functionally identical components.

The local database LS-D can be structured like the database FZ-D and in particular contain a plurality of stored sets of control commands G-SBS that are linked to procedure information VI. The local database LS-D can be embodied as a centralized or decentralized memory facility. The local database LS-D can in particular be part of a server system.

In the embodiment in FIG. 2, the management of the local database LS-D, and possibly the assignment of a remote operator, likewise takes place from the remote access facility 10. In particular, the local database LS-D can be queried and managed by the computing unit 11. In addition, sets of control commands G-SBS, F-SBS can be provided by the central database FZ-D or based on input from a remote operator.

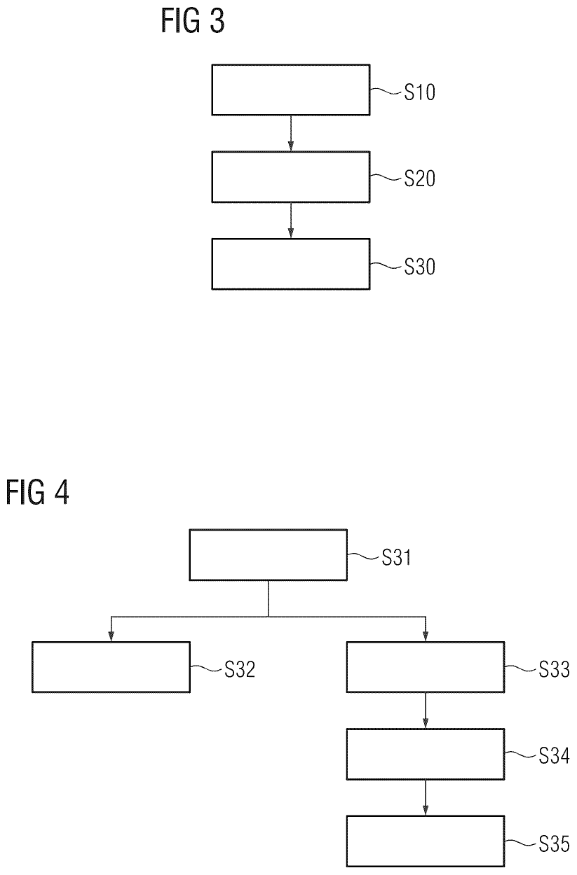

FIG. 3 depicts a schematic flowchart of a method for providing a set of control commands G-SBS, F-SBS for the performance of a medical imaging procedure according to one embodiment. The sequence of method steps is not restricted by either the sequence depicted or the selected numbering. For example, if necessary, the sequence of steps may be reversed and individual steps may be omitted.

First, in step S10 the database LS-D, FZ-D is provided. This can, for example, involve the database LS-D, FZ-D being constructed by the incorporation of sets of control commands G-SBS. The provision of the database LS-D, FZ-D can furthermore include the database LS-D, FZ-D being made available for querying data and in particular bringing it into data communication with the computing unit 11.

In step S20, a request REQ is received from one of the attached medical facilities 20, 30 via the communication interface 12 and acquired in the remote access facility 10. As explained, the request REQ is directed at the performance of an imaging procedure. The request REQ includes target procedure information Z-VI identifying the imaging procedure and optionally a time window in which the imaging procedure is to be executed.

Step S20 can including generating the request by the medical facility 20, 30 and transmitting the request from the medical facility 20, 30 to the remote access facility 10.

In step S30, sets of control commands F-SBS, G-SBS are provided in accordance with the request REQ. The provision in step S30 can in particular include transmitting the provided sets of control commands F-SBS, G-SBS to the medical facility 20, 30 via the communication interface 12.

Three alternative or mutually complementary ways are provided for how these sets of control commands F-SBS, G-SBS can be taught. First: sets of control commands G-SBS stored in the database FZ-D can be used. Second: a remote operator can be assigned to the imaging procedure to create a suitable set of control commands F-SBS by inputting it into a remote access workstation 13. In addition, sets of control commands can be generated using a trained function embodied to generate a set of control commands based on procedure information VI, Z-VI.

Step S30 can be followed by the transmission of the provided set of control commands F-SBS, G-SBS from the remote access facility 10 to the respective medical facility 20, 30 and/or controlling the respective imaging systems 21, 31 based on the transmitted set of control commands G-SBS, F-SBS in the respective medical facility 20.

FIG. 4 depicts a schematic flowchart of optional substeps of a method for providing a set of control commands F-SBS, G-SBS for the performance of a medical imaging procedure according to one embodiment. The sequence of method steps is not restricted by either the sequence depicted or the selected numbering. For example, if necessary, the sequence of steps may be reversed and individual steps may be omitted. The optional steps shown in FIG. 4 describe how sets of control commands F-SBS, G-SBS can be provided. In particular, the steps shown in FIG. 4 can be part of or precede step S30 in FIG. 3.

In step S31, first a check is performed as to whether the database FZ-D (or possibly the local database LS-D) contains a set of control commands G-SBS suitable for the imaging procedure to be performed. For this purpose, the target procedure information Z-VI can be compared with the procedure information VI stored in the database FZ-D, LS-D for the.

If the check in step S32 reveals that a suitable set of control commands G-SBS is already available, this can be retrieved from the database FZ-D, LS-D in step S32 and thus provided.

However, if the check in step S32 reveals that the database FZ-D, LS-D cannot be used for the request REQ, step S33 provides for determining a remote operator who can at least partially control the imaging procedure to be performed remotely.

Then, in step S34, input from the remote operator directed at the remote control of the imaging procedure is acquired as a set of remote control commands F-SBS. The input can, for example, be made into a remote access workstation 13. Herein, the remote operator input can be directed at direct control of the imaging systems 21, 31 and/or include instructions to local operators who then in turn operate the imaging system 21, 31 in compliance with the instructions.

Finally, in step S35, the set of remote control commands F-SBS input by the remote operator is provided. It should be noted that the provision of sets of control commands F-SBS can take place continuously while they are input and it is not necessary to wait until an imaging procedure has been completely processed by the remote operator before provision and transmission.

FIG. 5 depicts a schematic flowchart of optional substeps of a method for providing a set of control commands for the performance of a medical imaging procedure according to one embodiment. The sequence of method steps is not restricted by either the sequence depicted or the selected numbering. For example, if necessary, the sequence of steps may be reversed and individual steps may be omitted.

Herein, FIG. 5 depicts by way of example how the database FZ-D, LS-D can be constructed and/or further supplemented. Herein, it is assumed that the provided sets of control commands F-SBS, G-SBS contain at least one visual and/or audio-based instruction to a local operator. In particular, the steps depicted in FIG. 5 can follow step S30.

In a first step S40, operator actions performed locally on the imaging systems 21, 31 are recorded. Herein, the operator actions are directed at implementing instructions contained in the provided sets of control commands F-SGS, G-SBS. The operator actions can, for example, be recorded in the form of the steps and functions actually executed by the imaging system 21, 31. The operator actions are transmitted to the remote control facility 10.

In an optional substep S41, it can be assessed in the remote control facility 10 whether the operator actions performed meet a prespecified quality criterion. For example, it can be checked in step S41 whether a time specification has been adhered to. Furthermore, it can be checked in step S41 whether the operator actions included or necessitated corrections. Furthermore, it can be checked in step S41 whether the image data created by the operator actions is of sufficient quality. According to some embodiments, the subsequent steps S50 and S60 are only executed if the prespecified quality criterion is met.

In step S50, in the remote control facility 10, the operator actions are transferred to at least one further set of control commands with which the imaging system 21, 31 can preferably be actuated directly in future—without requiring the involvement of a local operator.

In step S60, the further set of control commands is stored in the database FZ-D, LS-D. Herein, the further set of control commands can be linked to the underlying target procedure information Z-VI.

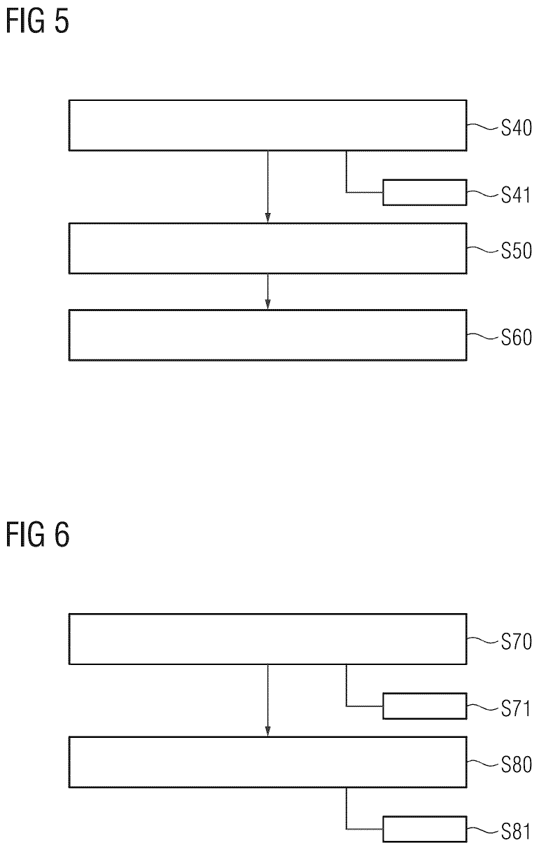

FIG. 6 depicts a schematic flowchart of optional substeps of a method for providing a set of control commands for the performance of a medical imaging procedure according to one embodiment. The sequence of method steps is not restricted by either the sequence depicted or the selected numbering. For example, if necessary, the sequence of steps may be reversed and individual steps may be omitted.

Herein, FIG. 6 is directed at taking account of the complexity level of the imaging procedure to be performed for the provision of sets of control commands F-SBS, G-SBS. The method steps depicted in FIG. 6 can be part of or precede step S30 in FIG. 3.

In step S70, first, at least one complexity level of the imaging procedure to be performed is determined. If the imaging procedure includes a plurality of procedure steps, herein, in step S71, a complexity level can be determined for each procedure step.

Complexity levels can, for example, already be transmitted with the request REQ and, for example, contained in the target procedure information Z-VI. Alternatively, such complexity levels can be determined based on the target procedure information Z-VI. For example, it is possible to use a predetermined classification of different imaging procedures according to their complexity level.

In step S80, the complexity level ascertained is compared with one or more prespecified threshold values. If the imaging procedure includes a plurality of procedure steps, such threshold value comparisons can take place for each procedure step (optional substep S81).

The threshold value comparisons can be used as a basis for further optimizing the provision of the sets of control commands G-SBS, F-SBS. For example, if the complexity level of the imaging procedure is above a prespecified threshold value, it may be determined that it is necessary to allocate a remote operator. Consequently, the set of control commands is provided as a set of remote control commands F-SBS and not as a stored set of control commands G-SBS. Conversely, it may be decided that the imaging procedure based on the complexity level should be considered so trivial that it can be performed by a local operator without the provision of sets of control commands.

If the imaging procedure includes a plurality of different procedure steps, it can be ascertained for each procedure step whether this step is sufficiently complex to require assistance by the provision of sets of control commands F-SBS, G-SBS. This enables the identification of trivial procedure steps for which no sets of control commands F-SBS, G-SBS need to be provided.

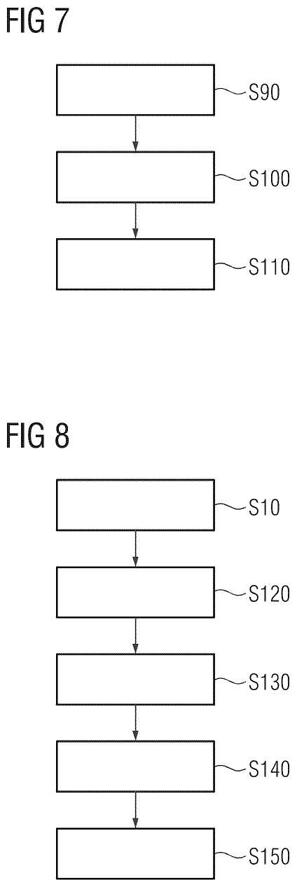

FIG. 7 depicts a schematic flowchart of optional substeps of a method for providing a set of control commands for the performance of a medical imaging procedure according to one embodiment. The sequence of method steps is not restricted by either the sequence depicted or the selected numbering. For example, if necessary, the sequence of steps may be reversed and individual steps may be omitted.

Herein, FIG. 7 is directed at retrospectively checking the quality of the provided sets of control commands F-SBS, G-SBS on the basis of the image data generated in the imaging procedure in order further to improve the sets of control commands F-SBS, G-SBS on this basis. The method steps depicted in FIG. 7 can be part of or follow step S30 in FIG. 3.

In step S90, the medical image data set generated by way of the imaging procedure performed with the provided sets of control commands F-SBS, G-SBS is received. Herein, the image data set can be acquired in the medical facility 20, 30, for example in the computing unit 24, 34 thereof. Furthermore, the image data set can be transmitted to the remote access facility 10 via corresponding communication interfaces 12, 26, 36 and acquired for example in the computing unit 11 thereof.

In step S100, the quality of the image data set is determined. For this purpose, the image information contained in the image data set can, for example, be assessed. In particular, it is possible to determine whether the body part to be mapped and the components thereof are completely mapped and have good resolution. Furthermore, values, such as contrast, sharpness, image homogeneity, image noise etc., can be read from the image data set.

Then, in step S110, the quality ascertained can be used as the basis for adapting the previously provided set of control commands G-SBS. For example, settings for the imaging system 21, 31 contained in the set of control commands G-SBS, F-SBS can be adapted or optimized in order to improve the quality the next time the set of control commands G-SBS is retrieved.

FIG. 8 depicts a schematic flowchart of optional steps in a method for providing a set of control commands for the performance of a medical imaging procedure according to one embodiment. The sequence of method steps is not restricted by either the sequence depicted or the selected numbering. For example, if necessary, the sequence of steps may be reversed and individual steps may be omitted.

Herein, FIG. 8 is directed at additionally using the stored sets of control commands G-SBS for checking operator actions. In particular, this enables it to be ascertained whether an operator action conforms to a set of control commands G-SBS. The method steps depicted in FIG. 8 can, for example, follow step S30 in FIG. 3. Alternatively, the method described in FIG. 8 can also be executed independently of the method described in FIG. 3.

In step S120, first, an operator action directed at the performance of an imaging procedure is received from the medical facility 20, 30. Then, the operator action can be transmitted to the remote control facility 10 together with procedure information VI identifying the imaging procedure.

In step S130, the remote control facility 10 retrieves a reference or comparison set of control commands G-SBS from the database FZ-D, LS-D. This can again be based on a comparison of the procedure information VI transmitted in step S120 with the procedure information VI stored in the database FZ-D, LS-D.

In step S140, the operator action is assessed based on the comparison set of control commands G-SBS. For this purpose, the operator action can be compared with the comparison set of control commands G-SBS. In particular, the operator action can be checked for conformity with the comparison set of control commands G-SBS. Furthermore, an efficiency value of the operator action can be ascertained based on the comparison with the comparison set of control commands G-SBS.

In step S150, the assessment outcome from step S140 is provided. This can in particular include transmitting the assessment outcome to the medical facility 20, 30 in order to provide feedback on operator actions as a further service.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Where this has not yet been done explicitly, but makes sense and is in the spirit of the present invention, individual exemplary embodiments, individual partial aspects or features thereof can be combined with one another or exchanged without departing from the scope of the present invention. Where applicable, advantages of the present invention described with reference to one exemplary embodiment also apply to other exemplary embodiments without explicit mention.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for providing a set of control commands for controlling a medical imaging system in a medical facility, the computer-implemented method comprising:
providing a database storing at least one set of control commands for controlling medical imaging systems when performing medical imaging procedures;
acquiring, by a remote access facility, a request from the medical facility for the performance of a medical imaging procedure with the medical imaging system, wherein said request includes target procedure information describing the medical imaging procedure to be performed; and
providing, by the remote access facility, a set of control commands to the medical facility, the set of control commands based on a query of the database using the target procedure information.

2. The computer-implemented method as claimed in claim 1,
wherein the database is arranged in the remote access facility.

3. The computer-implemented method as claimed in claim 1, wherein the providing of the set of control commands includes:
querying, by the remote access facility based on the target procedure information, whether the medical imaging procedure to be performed can be performed with a set of control commands stored in the database;
in response to determining that the medical imaging procedure can be performed, providing the stored at least one set of control commands as the set of control commands to the medical facility; and
in response to determining that the medical imaging procedure cannot be performed, providing the set of control commands by
assigning a remote operator in the remote access facility for remote control of the medical imaging procedure to be performed by the remote operator,
acquiring a set of remote control commands input to the remote access facility by the remote operator for the performance of the medical imaging procedure to be performed, and
providing, by the remote access facility, the set of remote control commands input to the remote access facility as the set of control commands to the medical facility.

4. The computer-implemented method as claimed in claim 3, wherein
the set of remote control commands induce, in the medical facility, at least one of at least one visual-based instruction or at least one audio-based instruction for an operator for the performance the medical imaging procedure to be performed; and
the computer-implemented method further includes
logging at least one operator action performed by the operator based on the set of remote control commands, on the medical imaging system,
determining, by the remote access facility, at least one further set of control commands based on the at least one operator action, and
storing, by the remote access facility, the at least one further set of control commands in the database.

5. The computer-implemented method as claimed in claim 4, further comprising:
ascertaining a quality measure of the at least one operator action by the remote access facility, wherein the determining and the storing of the at least one further set of control commands are based on the ascertaining.

6. The computer-implemented method as claimed in claim 3, further comprising:
determining a complexity level of the medical imaging procedure to be performed based on the target procedure information;
comparing the complexity level with a threshold value; and wherein
in response to the complexity level being above the threshold value, the set of control commands is provided as the set of remote control commands and not as a stored set of control commands.

7. The computer-implemented method as claimed in claim 1, further comprising:
receiving a medical image data set as the outcome of the medical imaging procedure; and
adapting the set of control commands provided to the medical facility based on the medical image data set.

8. The computer-implemented method as claimed in claim 1, wherein
the medical imaging procedure to be performed includes a plurality of different procedure steps; and
the computer-implemented method further includes
determining a complexity level for each procedure step based on the target procedure information, and
for each respective procedure step, ascertaining whether the complexity level exceeds a threshold value, and wherein
for the respective procedure step, the set of control commands is provided only in response to the complexity level of the respective procedure step exceeding the threshold value.

9. The computer-implemented method as claimed in claim 1, further comprising:
receiving an operator action in the medical facility when performing an imaging procedure and receiving procedure information describing the imaging procedure;
ascertaining a comparison control command dataset by querying the database based on the procedure information describing the imaging procedure;
determining an efficiency value of the operator action based on a comparison between the operator action and the comparison control command dataset by the remote access facility; and
providing the efficiency value.

10. The computer-implemented method as claimed in claim 1,
wherein the database is arranged in the medical facility and stores sets of control commands specific to the medical facility.

11. The computer-implemented method as claimed in claim 1, wherein the target procedure information includes one or more of:
an element specifying a body part of a patient to be mapped with the medical imaging procedure,
an element specifying a diagnostic context of the medical imaging procedure,
an element specifying different individual procedure steps to be performed within the medical imaging procedure,
an element specifying the medical imaging system to be used within the medical imaging procedure, and
an element specifying a complexity level of at least one of the medical imaging procedure or individual procedure steps of the medical imaging procedure.

12. The computer-implemented method as claimed in claim 1, wherein the medical imaging system includes one or more of:
a medical imaging device,
a radiology information system,
a picture archiving and communication system,
a treatment scheduling system,
a patient positioning system, or
an injection device for substance administration to a patient.

13. A facility for providing a set of control commands for controlling a medical imaging system arranged in a medical facility, wherein the facility comprises:
a communication interface configured to communicate with the medical facility;
a database storing at least one set of control commands for controlling medical imaging systems when performing medical imaging procedures; and
a computing unit configured to
acquire, via the communication interface, a request for performance of a medical imaging procedure with the medical imaging system from the medical facility, wherein the request includes target procedure information describing the medical imaging procedure to be performed, and
provide a set of control commands usable for the medical imaging procedure to be performed, the set of control commands being provided based on a querying of the database using the target procedure information.

14. A non-transitory computer-readable storage medium storing computer-executable instructions that, when executed by at least one processor at a device for providing a set of control commands for controlling a medical imaging system, cause the device to perform the computer-implemented method of claim 1.

15. The computer-implemented method as claimed in claim 2, wherein the database is arranged outside the medical facility.

16. The computer-implemented method as claimed in claim 5, wherein the determining and the storing of the at least one further set of control commands are performed only when a quality measure meets a quality criterion.

17. The computer-implemented method as claimed in claim 7, wherein the adapting comprises at least one of:
adapting at least one set of control commands stored in the database; or
adding a further set of control commands to the database based on the adapted set of control commands.

18. The computer-implemented method as claimed in claim 12, wherein the medical imaging device is one of an X-ray device, a computed tomography device, a magnetic resonance imaging device, a positron emission tomography device, an ultrasound device or a radiotherapy device.

19. The computer-implemented method as claimed in claim 12, the injection device is a contrast medium injector.

20. The computer-implemented method as claimed in claim 3, wherein
the medical imaging procedure to be performed includes a plurality of different procedure steps; and
the computer-implemented method further includes
determining a complexity level for each procedure step based on the target procedure information, and
for each respective procedure step, ascertaining whether the complexity level exceeds a threshold value, and wherein
for the respective procedure step, the set of control commands is provided only in response to the complexity level of the respective procedure step exceeding the threshold value.
